(19) United States
(12) Patent Application Publication
(10) Pub. No.: US 2015/0359802 A1
(43) Pub. Date: Dec. 17, 2015

(54) PARENTERAL PHARMACEUTICAL FORM WHICH RELEASES AROMATSE INHIBITOR AND GESTAGENS, FOR THE TREATMENT OF ENDOMETRIOSIS

(71) Applicant: Bayer Intellectual Property GmbH, Monheim (DE)

(72) Inventors: Arto PAKKALIN, Helsinki (FI); Rudolf KNAUTHE, Glienicke (DE); Heinz SCHMITZ, Berlin (DE); Christine TALLING, Turku (FI); Harri JUKARAINEN, Kuusisto (FI); Henriikka KOROLAINEN, Loimaa (FI)

(21) Appl. No.: 14/743,935

(22) Filed: Jun. 18, 2015

Related U.S. Application Data
(63) Continuation of application No. 13/638,243, filed on Jan. 3, 2013, now abandoned.

(30) Foreign Application Priority Data
Mar. 31, 2010 (DE) 102010003494.0

(51) Int. Cl. A61K 31/567; A61K 9/00; A61K 31/4196

(52) U.S. Cl. CPC A61K 31/567; A61K 31/4196; A61K 9/0036

(57) ABSTRACT

The present invention for the treatment of endometriosis relates to providing a parenteral dosage form (delivery system) for the controlled release of an aromatase inhibitor (AI) in a daily release rate that does not induce stimulation of the ovaries by negative feed-back of the pituitary-ovarian-axis (which would cause secretion of gonadotropins and stimulation of ovarian follicular growth) and a gestagen in a daily release rate below the ovulation inhibition dose that provides contraceptive efficacy based on local effects (e.g. reducing and thickening of the cervical mucus impairing sperm ascension, effects on the endometrium and on tubal motility impairing implantation and egg transport).

The present invention for the treatment of endometriosis relates to providing a parenteral dosage form (delivery system) for the controlled release of an aromatase inhibitor (AI) at a rate that does not induce stimulation of the ovaries by negative feed-back of the pituitary-ovarian-axis (no increase in the secretion of gonadotropins which would induce stimulation of follicular growth) and a gestagen (progestin/progestogen) at a rate that provides contraceptive efficacy based on known local effects (such as e.g. reduction and thickening of the cervical mucus impairing sperm ascension, effects on the endometrium and on tubal motility impairing implantation and egg transport). The combination of an AI and a gestagen at an ovulation-inhibiting dosage would result in estrogen-deficiency symptoms owing to strong suppression of endogenous estrogen synthesis (e.g. hot flushes, reduction in bone density). Owing to the low dosages used in this invention (A1 without counterregulation and gestagen without reliable inhibition of ovulation), the risk of estrogen-deficiency symptoms is effectively minimized by the combination. The preferred dosage form described here is a polymer-based dosage form that comprises at least one compartment, said one or each compartment comprising a core or a core encased by a membrane, the core and the membrane essentially consisting of the same or different polymer compositions, wherein at least one compartment comprises an AI and at least one compartment, which may be the same or different from the one comprising the AI, comprises a gestagen. The parenteral dosage form can be any dosage form suitable for delivering therapeutically active agents at a controlled release rate over a prolonged period of time (for example for an intravaginal ring [IVR; the terms intravaginal ring and vaginal ring are used synonymously]) 1 week to 3 months, preferably 4 to 6 weeks, for an intrauterine device (IUD, the terms intrauterine device and intrauterine system are used synonymously) this application time can be 3 months to 1 year or more. The preferred dosage form as said is either an IVR or an IUD which offers the additional advantage to achieve additional local effects at endometriotic lesions in the vicinity of the application site.

Endometriosis is a chronic disease affecting approx. 10% of women in reproductive age. The disease is characterized by the presence of endometrium-like tissue outside the uterine cavity. Various theories about the pathogenesis of endometriosis exist. Probably in most cases it is initiated by a retrograde menstruation in which endometrial tissue passes through the fallopian tubes into the abdominal cavity where endometrial cells adhere to the surfaces of the abdominal tissues and organs to form ectopic endometrial implants, i.e. endometriotic lesions. This endometrium-like tissue can respond in the same way as the normal endometrium to changes in the hormonal environment during the menstrual cycle so that as the concentrations of estrogen and progesterone change, the tissue reacts in the same way as the endometrium itself. However, in the course of the disease, these endometriotic lesions may uncouple from the normal menstrual cycle. The presence of endometrial implants on abdominal surfaces (endometrial nodules) can induce an inflammatory reaction which together with growth of nerve fibers may represent the pathophysiological/anatomic correlates causing symptoms typically associated with endometriosis such as pelvic pain, dysmenorrhea and dyspareunia.

Current treatments indicated for endometriosis are based on inhibition of ovarian estrogen production via central inhibition of the pituitary-ovarian-axis (e.g. gonadotropin releasing hormone-analogs (GnRH-analogs), danazol, medroxyprogesterone acetate, dienogest, combined oral contraceptives (COCs)). However, inhibition of ovarian estrogen production during treatment with GnRH analogs leads to side-effects related to estrogen deficiency like hot flushes and bone loss as the most relevant ones if no estrogen is added to the treatment. Other side-effects may comprise: transient vaginal bleeding, vaginal dryness, decreased libido, breast tenderness, insomnia, depression, irritability and fatigue, headache, and decreased elasticity of the skin. Therefore, to reduce these side effects during GnRH-analog therapy so-called add-back regimens were established in which (conjugated) estrogens or norethisterone acetate (NETA, which is metabolized partly to estradiol) were added to the therapy with GnRH-analogs. Both treatments (GnRH-analogs+estrogen or GnRH-analogs+NETA) are applied with their full effective dose which means also that the entire spectrum of expected side effects to these medications may occur. COCs applied on their own are effective in the treatment of endometriosis, too and do not require any add-back treatment.

However, as is also the case with add-back regimens, exogenous estrogen is applied to the patient by treatment with COCs, in this case the strong estrogen ethinylestradiol. In this case, the application of exogenous estrogen may theoretically impair efficacy of the gestagen or of the GnRH-analog against the estrogen-dependent disease endometriosis.

On the other hand inhibition of the pituitary-ovarian-axis has no influence on sites of estrogen production outside the ovaries which may be of crucial importance for new treatment modalities of endometriosis. Previous investigations have demonstrated that the enzyme aromatase, catalyzing the conversion of testosterone and other androgenic precursors to estrogen, is expressed within endometriotic lesions (Urabe M et al, Acta Endocrinol (Copenh). 1989, 121(2):259-64, Noble L S et al, J Clin Endocrinol Metab. 1996, 81(1):174-9). Consequently, and this may explain treatment failures to above-mentioned therapies which merely inhibit ovarian production of estrogen, endometriotic lesions can produce significant amounts of estradiol locally. Additionally, it has been shown that the inflammatory mediator Prostaglandin E2 acts as a potent stimulator of aromatase expression, further enhancing local estrogen production in the inflammatory milieu of endometriotic lesions (Noble L S et al, J Clin Endocrinol Metab. 1997, 82(2):600-6).

AIs in typical dosages (e.g. Anastrozole 1 mg/day) reduce systemic estrogen levels in post-menopausal women by more than 85% (Geisler J et al, J Clin Oncol 2002, 20(3): 751-757). In pre-menopausal women this effect is reduced by counterregulation via the pituitary-ovarian-axis (i.e. pituitary sensing of decreased systemic estrogen levels leads to consecutive secretion of gonadotropins which stimulate estrogen synthesis in the ovaries and partly overrule the effect of the AI), which results in stimulation of ovarian follicular growth (in fact, this effect is taken advantage of in patients suffering from ovarian subfertility to stimulate follicular growth). For this reason in endometriosis patients AIs had been used in dosages typically used in postmenopausal women to treat breast cancer in combination with drugs inhibiting counterregulation in various clinical trials, e.g. with NETA (Ailawadi R K et al, Fertility & Sterility 2004, 81(2): 290-296), or COCs (Amsterdam L L et al 2005, Fertility & Sterility 2005, 84(2): 300-304). In addition to inhibition of counterregulation, the reduction of side effects related to estrogen deficiency is seen as an advantage of these combinations. However, administration of exogenous estrogen or NETA in these combinations may reduce the efficacy (cf. above) of AIs with respect to treatment of symptoms of endometriosis.

WO 03/15872 describes a method of treating or preventing uterine fibroids or endometriosis by administering an AI to a patient intravaginally. The invention discloses the advantage of local effects of monotherapy with AIs claiming the reduction of systemic side effects by local administration. The application does not disclose the combination of an AI with a gestagen in the form of a parenteral dosage form and in particular not a combination of an AI with a gestagen in an IVR or IUD. In contrast to the present invention the WO 03/15872 does not disclose any means to achieve contraceptive efficacy, which is essential in this invention, as it is of crucial importance to a meaningful product profile to prevent pregnancy as long as a woman of childbearing age is under treatment with an AI. The technical solution as described in this invention is to combine both the AI and the contraceptive activity of a gestagen in one parenteral dosage form to avoid the physical separation of both and thereby exclude the possibility that an AI is used to treat endometriosis without a contraceptive protection. This possibility is not excluded when two physically separable dosage forms are used.

The combination of an AI with a gestagen (AI+NETA, Ailawadi R K et al 2004) or a COC (Amsterdam L L et al 2005; WO 04/69260) for oral use has been suggested as well. Both combinations aim to prevent estrogen deficiency symptoms by exogenous administration of estrogenic activity (estrogen metabolism of NETA; ethinylestradiol in COCs). The disadvantage of these treatment modalities and the differentiation to the invention described in this application is that in both cases administration of exogenous estrogen activity (NETA is partially converted into estrogens; COCs contain the strong estrogen ethinylestradiol) is necessary to avoid side effects. This, however, attenuates the pharmacodynamic effect of the AI on endometriotic tissue. Furthermore, these disclosures do not describe the advantages of a local application of the AI inhibiting the locally expressed aromatase of endometrial lesions in the vicinity of the dosage form and thereby reducing the dose needed to achieve the desired full pharmacological effect.

Closest to the invention described in this application may be the patent application WO 03/17973 which discloses the application of AIs via the vaginal route, alone or in combination with other estrogen metabolism-influencing compounds, e.g. cyclooxygenase-2 inhibitors (COX 2 inhibitors), 17-beta-hydroxy-steroid-dehydrogenase-1 inhibitors (17βHSD-1 inhibitors). Further the invention claims a method that does not inhibit ovarian estrogen synthesis. The invention discloses the advantage of combinations of AIs with other estrogen metabolism-influencing drugs via local application. The application does not disclose the combination of an AI with a gestagen in the form of a parenteral dosage form and in particular not a combination of an AI with a gestagen in the described dosing in an IVR. In contrast to the present invention WO 03/17973 does not disclose any means to achieve contraceptive efficacy. Again it is important to recognize that only the physical not separable combination of the AI activity and the contraceptive effect leads to a meaningful product.

US 2011/0033519 A1 (publication date: Feb. 10, 2011) describes dosage forms which deliver aromatase inhibitors, optionally in combination with contraceptive substances, locally into uterine tissue. Thereby, diseases such as myomas, adenomyosis and endometriosis shall be treated or prevented. Since gestagens might stimulate the growth of myomas, their use is not advised and, instead, copper and other noble metals are preferred as the basis of contraception. Suitable IUD aromatase inhibitor doses are—for example for anastrozole—reported to be 1 μg to 10 mg per day. However, the patent proposes a period of use of 5-10 years, which appears to be hardly feasible from a technical point of view.

One aspect of the invention described in this application when using an IVR/IUD is based on the concept to apply a dose of AI locally which does not induce counterregulatory effects of the pituitary-ovarian-axis but exhibits its aromatase inhibitory effect in the endometriotic lesions. Without counterregulatory effects as a consequence of AI administration, there is no need to apply a gestagen or a COC for inhibition of the pituitary-ovarian-axis which enables dose reduction of the gestagen to the dose necessary to achieve contraceptive efficacy by local mechanisms. In this manner estrogen deficiency symptoms will be avoided and no exogeneous estrogen administration will be necessary. Furthermore, as endometriosis is an estrogen dependent disease the absence of administration of exogenous estrogens does not impair the therapeutic efficacy of the AI. Since gestagens also have an inhibitory effect on aromatase expression, the gestagen in this invention could add to the effect of the AI.

To avoid counterregulatory effects of the pituitary-ovarian-axis with the highest possible dose of AI on the one hand and to achieve best contraceptive efficacy of the gestagen-only based contraception with the highest possible dose of gestagen below the ovulation inhibition dose on the other, it is necessary to apply the active ingredients in a formulation with controlled release avoiding high fluctuations of serum levels which could trigger counterregulation by the pituitary-ovarian-axis. This will be achieved by a parenteral dosage form, preferably an IVR or IUD.

In this manner the invention described in this application combines an effective treatment of endometriosis with a reliable contraceptive method in an application mode supporting high compliance by a parenteral dosage form (no AI intake without contraceptive protection, therefore no unwanted exposure of an embryo to an AI). In contrast to the methods described in the state of the art the combination in this invention will reduce the drug exposure of both, the AI and the gestagen to the amount necessary for efficacy which will also minimize the risk for unfavorable side-effects associated with diminished estrogen levels like e.g. hot flushes, bone loss etc.

In order to minimise the risk of estrogen deficiency-related side effects, the gestagen exposure sought in this invention will be below the exposure achieved by administration of a given gestagen in ovulation inhibition dose (irrespective of route of administration), but high enough to provide contraceptive efficacy by local effects as measured e.g. by the Insler score (Insler V et al, Int J Gynecol Obstet 1972, 10: 223-228). The oral ovulation inhibition dose of various gestagens—which, after oral administration, lead to particular gestagen-specific plasma or serum concentrations—are described in the literature as e.g. in Neumann F et al, Reproduktionsmedizin 1998, 14: 257-264 or Taubert H D, Kuhl, H, Kontrazeption mit Hormonen, 2. Aufl. 1995. More specifically: The AI dose in the combination will not substantially stimulate ovarian activity beyond the typical gestagen-only effect as expected for this invention in the dose of gestagen to be administered. The experimental setup to determine the dose of the gestagen and AI is described in the experimental part.

The dosage form according to the invention comprising a combination of an AI and a gestagen is especially suitable for the treatment of endometriosis providing efficacy against symptoms related to endometriosis minimising the risk of side-effects related to estrogen-deficiency (e.g. bone loss, hot flushes). At the same time the invention will provide a physically not separable daily exposure to a gestagen to ensure a reliable contraceptive efficacy and thus avoid any risk of pregnancy with subsequently the unwanted exposure of an embryo to an AI. This is a major aspect of the invention as it improves the safety of the desired product meaningfully (see by way of contrast WO 03/15872 and WO 03/17973). Furthermore, in contrast to oral application, the parenteral/local application in a dosage form with a controlled release rate as e.g. realised with the preferred solution (IVR/IUD) allows for dosing appropriate to achieve the desired medical outcome with best possible reduction of major side effects related to fluctuating exposure of the active ingredients (amplitude between maximum serum levels after e.g. intake of oral formulations and trough serum levels before next intake). Additionally, the local application may be especially advantageous for treatment of endometriotic lesions in the vicinity of the parenteral dosage form (e.g. in the case of vaginal endometriosis, deep infiltrating endometriosis, adenomyosis or endometriosis of the cul-de-sac).

Aromatase inhibitors are compounds that inhibit the action of the enzyme aromatase, which converts androgens into estrogens by a process called aromatization. By their action AI reduce or block the synthesis of estrogens. Selective AI are e.g. anastrozole (Arimidex®), exemestane (Aromasin®), fadrozole (Afema®), formestane (Lentaron®), letrozole (Femara), pentrozole, vorozole (Rivizor®) or the AI BGS649 from Novartis which, to date, can be found in clinical development (clinicaltrials.gov-Identifier: NCT01116440; NCT01190475) and pharmaceutical acceptable salts thereof.

A parenteral dosage form is a dosage form for administration of drugs in which absorption of the drugs takes place via circumvention of the gastrointestinal tract. It can be any dosage form suitable for delivering therapeutically active agents at a controlled release rate over a prolonged period of time. Thus, the dosage form can be formulated in a wide variety of applications including for example transdermal patches, implants, depot injections (including microparticles, in situ depot forming dosage forms etc.), intravaginal, intracervical and intrauterine dosage forms. According to a preferred embodiment, the dosage form is an IVR, or an IUD. An IVR is a substantially ring-shaped polymeric dosage form which provides controlled release of active ingredient(s) to the vagina over extended periods of time. An IUD is any polymeric dosage form which provides controlled release of active ingredient(s) intrauterine to the uterus over extended periods of time. A subcutaneous implant is a substantially rod-shaped polymeric dosage form comprising one or more rods which provides controlled systemic release of active ingredient(s) to the body over extended periods of time.

Release rate means the mean, released amount of active drug substance in 24 hours from the dosage form that is available for absorption by the surrounding tissue. A person skilled in the art will know that the mean release rate from a parenteral dosage form can decrease over the period of application.

A controlled long-term release dosage form means any dosage form suitable for administration of drugs over a prolonged period of time avoiding fluctuations of drug levels normally induced by immediate release formulations (e.g. tablets, injections, etc.).

A gestagen is a synthetic progestogen that has progestogenic effects similar to progesterone. Gestagens other than progesterone are e.g. allylestrenol, chlormadinone acetate, cyproterone acetate, desogestrel, dienogest, drospirenone, dydrogesterone, etonogestrel, ethynodiol, gestodene, levonorgestrel, lynestrenol, medrogestone, medroxyprogesterone, megestrol acetate, nomegestrol, norethindrone, norethisterone, norethynodrel, norgestimate, norgestrel, quingestrone or trimegestone and other approved or commercially available gestagens, and pharmaceutical acceptable salts thereof. These gestagens can also be provided as esters or any other suitable chemical modifications.

A gestagen in a daily release rate below the ovulation inhibition dose but high enough to provide reliable contraceptive protection means that known effects as e.g. reduction and thickening of the cervical mucus impairing sperm ascension, effects on the endometrium and on tubal motility impairing implantation and egg transport prevent fertilization of an ovum. A gestagen dosage which is typical for this effect is to be found in the preparation Microlut® with a tablet dosage of 30 μg of levonorgestrol.

Typical oral ovulation inhibition doses are (Neumann F et al, Reproduktionsmedizin, 1998, 14: 257-264; Taubert H D, Kuhl, H, Kontrazeption mit Hormonen, 2. Aufl. 1995):

Ovulation inhibition dose
[μg/day p.o.]

| Gestagen | Neumann et al | Taubert &Kuhl |
|---|---|---|
| Norethisterone | 500 | 400 |
| Norethisterone acetate | 500 | |
| Lynestrenol | 2000 | 2000 |
| Norgestimate | 200 | 200 |
| Levonorgestrel | 50 | 60 |
| Desogestrel | 60 | 60 |
| Gestodene | 30 | 30 |
| Dienogest | 1000 | |
| Chlormanidone acetate | 1500-2000 | 1700 |
| Cyproterone acetate | 1000 | 1000 |
| Medroxyprogesterone | 10 | |
| acetate | | |
| Drospirenone | 2000 | |
| 3-Keto-Desogestrel | | 60 |

Note:
To a person skilled in the art it is known that values for the ovulation inhibition dose of gestagens vary to a certain degree due to methodological and statistical reasons. The gestagen dose/exposure used in this invention will be below the exposure which would lead to reliable ovulation inhibition in the case of parenteral or oral application. For oral applications the ovulation inhibition dose is given in the literature and as example in the table above.

If the dose which inhibits ovulation is not known for a given gestagen, the release rate to be used for a parenteral dosage form will be determined in a pharmacokinetic/pharmacodynamic study in which the ovarian, cervical, and hormonal effects of different dosages of a gestagen to be used will be measured (ovarian activity by transvaginal ultrasound, hormone levels in blood, Insler score on the cervical mucus). As an example of an ovulation-inhibiting dose which is not certain but locally effective, systemic exposure of levonorgestrel (LNG) after release from the IVR corresponds to exposure of levonorgestrel after oral administration in a daily dosage which is higher than 10 μg but lower than 50 μg.

A considerably increased potential release of active ingredients shortly after insertion (so called burst effect) is known to a person skilled in the art from IVR, IUD or polymer based implants. IVR, IUD and polymer based implants showing such a burst effect shortly after insertion are also considered to be claimed even if during the duration of the burst effect the release rate is increased.

An aromatase inhibitor (AI) in a daily release rate that does not induce stimulation of the ovaries by negative feed-back of the pituitary-ovarian-axis (no increase in the secretion of gonadotropins which would induce stimulation of follicular growth) means the highest dose which does not induce additional follicular growth as compared to the gestagen-treated cycle as investigated by determination of blood hormone levels (follicle stimulating hormone=FSH, luteinizing hormone=LH, estradiol, progesterone) and transvaginal ultrasound measurements.

If not known for a given AI, the release rate to be used for a parenteral dosage form will be determined according to example 2 of this application. For anastrozole, the systemic exposure achieved by the dosage form is on average less than the exposure produced by 1 mg (or between 0.1 mg and 0.9 mg) per day/orally. For letrozole, the systemic exposure achieved by the dosage form is less than the exposure produced by 2.5 mg (or between 0.1 mg and 2.4 mg) per day/orally. Pharmacokinetic accumulation phenomena should be considered here.

A considerably increased potential release of active ingredients shortly after insertion (so called burst effect) is known to a person skilled in the art from IVR, IUD or polymer based implants. IVR, IUD and polymer based implants showing such a burst effect shortly after insertion are considered to be claimed even if during the duration of the burst effect the release rate is increased.

The application in an IVR provides a convenient formulation with low variability in drug serum levels, avoiding hepatic first-pass metabolism of the drug substance and improving treatment compliance since no daily remembering of drug intake is required. In particular, the contraceptive principle of the gestagen pill (POP, “progestin only pill”) in a dosage below the ovulation inhibition dose would require an exact dosing schedule to ensure a reliable contraceptive effect. In that aspect the continuous administration with an IVR is of great advantage. The local application allows for dosing appropriate to achieve the desired medical outcome with reduction of major side effects related to systemic exposure of the active ingredients. To a person skilled in the art, it is known that application of an IVR (or alternative depot formulations, more particularly in the case of polymer-based dosage forms as well) can lead to a change (decrease) in the daily release rate over the period of administration. Dosage forms which exhibit such a change are considered to be claimed. Preferred dosage forms are dosage forms for local application, more particularly IVRs and IUDs. An IVR is particularly preferred.

Preferred IVRs and IUDs contain anastrozole as aromatase inhibitor. Particular preference is given to an anastrozole-containing IVR. Particular preference is likewise given to an anastrozole-containing IVR in which the systemic anastrozole exposure achieved after release from the IVR corresponds to the anastrozole exposure after oral administration in a dosage of less than 1 mg (or between 0.1 mg and 0.9 mg) of anastrozole per day. Likewise, it is particularly preferred for this IVR to contain levonorgestrel as gestagen.

Preferred IVRs and IUDs contain levonorgestrel, dienogest or gestodene as gestagen. Particular preference is given to an IVR having levonorgestrel as gestagen. Particular preference is likewise given to an IVR in which the systemic levonorgestrel exposure achieved after release from the IVR corresponds to the levonorgestrel exposure after oral administration in a dosage of more than 10 μg, but less than 50 μg, per day. Likewise, it is particulary preferred for this IVR to contain anastrozole as aromatase inhibitor.

Very particular preference is given to an IVR having anastrozole as aromatase inhibitor and levonorgestrel as gestagen. Very particular preference is likewise given to an IVR which contains both anastrozole as aromatase inhibitor and levonorgestrel as gestagen and in which the systemic anastrozole exposure achieved after release from the IVR corresponds to the anastrozole exposure after oral administration in a dosage of less than 1 mg (or between 0.1 mg and 0.9 mg) of anastrozole per day and in which the systemic levonorgestrel exposure achieved after release from the IVR corresponds to the levonorgestrel exposure after oral administration in a dosage of more than 10 μg, but less than 50 μg, per day.

For the particularly preferred IVR, the duration of the long-term release is from one week to three months, particularly preferably from 4 to 6 weeks. For the likewise preferred IUD, the long-term release is at least 3 months, preferably one year or longer.

Owing to the burst effect, the dosage forms according to the invention may achieve the desired release rates according to the invention only one, two or three days after the start of treatment, in exceptional cases only after a week. The start of treatment means here the time at which the dosage form is applied.

All the preferred embodiments mentioned here can be used for treating endometriosis. Particular preference is given to the treatment of endometriosis with simultaneous contraception. Particular preference is likewise given to a method for simultaneous treatment of endometriosis and for contraception using, as the case may be, one of the abovementioned preferred dosage forms.

### DETAILED DESCRIPTION OF A PARENTERAL DOSAGE FORM

Parenteral dosage forms, including for example implants, intrauterine devices and intravaginal rings, capable of providing controlled release of active ingredient(s) over extended periods of time, are typically formed from biocompatible polymers and contain a drug or drugs released by diffusion through the polymer matrix. A number of different constructions of the dosage forms are known from the literature. Some dosage forms may comprise a polymer matrix but no membrane or wall encasing said matrix (monolithic dosage form), whereas some other dosage forms comprise a polymer matrix, a core, encased by a membrane. Extensive use has been made of the simultaneous administration of two or more therapeutically active substances, and a number of different constructions of the dosage forms are known from the literature.

According to an embodiment of the invention, the dosage form comprises at least one compartment comprising a core, or a core encased by a membrane, said core and membrane comprising the same or different polymer composition, wherein at least one of said compartments comprises an AI, and optionally at least one compartment, which may be the same or different from the one comprising the AI, may comprise a gestagen or a compound having a progestogenic activity.

Thus the compartment comprises essentially a polymer composition wherein the polymer composition of the core, of the membrane or of both may comprise a therapeutically active substance or substances. The polymer composition can be suitably chosen so that the release of the therapeutically active agent is regulated by the core, the membrane or both.

According to the embodiment in which the dosage form comprises two or more compartments, said compartments may be positioned next to each other, side-by-side, one on the other or be at least partly within each other, and may further be separated from each other by a separation membrane or by an inert placebo compartment. Compartments may be solid or hollow.

The membrane, if any, may cover the whole dosage form or cover only a part of the dosage form, whereby the degree of extension can vary depending on a number of factors, for example such as the choice of materials and the choice of active agents. The membrane may consist of more than one layer. The thickness of the membrane depends on materials and active agents used as well as on desired release profiles, but generally the thickness is smaller than the thickness of the core member.

Polymer compositions of the core, the membrane and the possible separation membrane or the inert placebo compartment, can be the same or different and may stand for one single polymer or a mixture of polymers or may be made up of polymers that are blended with each other.

In principle any polymer, either biodegradable or non-biodegradable, can be used as long as it is biocompatible. Examples of commonly used polymeric materials include, but are not limited to, polysiloxanes, polyurethanes, thermoplastic polyurethanes, ethylene/vinyl acetate copolymers (EVA), and copolymers of dimethylsiloxanes and methylvinylsiloxanes, biodegradable polymers, for example poly(hydroxyalkanoic acids), poly(lactic acids), poly(glycolic acids), poly(glycolides), poly(L-lactides), poly(lactide-co-glycolides), and a mixture of at least two of them.

The structural integrity of the material may be enhanced by the addition of a particulate material such as silica or diatomaceous earth. The polymer composition can also comprise additional material for example to adjust hydrophilic or hydrophobic properties in order to achieve the desired release rate of one or several of the therapeutic substances, while taking into account that all additives need to be biocompatible and harmless to the patient. The core or membrane may also comprise for example complex forming agents such as cyclodextrin derivatives to adjust the initial burst of the substance to the accepted or desired level. Auxiliary substances, for example such as tensides, anti-foaming agents, stabilizers, solubilisers or absorption retarders, or a mixture of any two or more of such substances, can also be added in order to impart the desired physical properties to the body of the dosage form. Further, additives such as pigments, glossing agents, matting agents, colorants, mica or equal can be added to the body of the dosage form or the membrane or to both in order to provide the dosage form with a desired visual appearance.

### Manufacture of a Parenteral Dosage Form

The parenteral dosage form according to this invention can be manufactured in accordance with standard techniques known in the art, and the shape and size of the dosage form may be freely chosen by the person skilled in the art.

A sufficient amount of at least one therapeutically active agent can be incorporated in the polymer composition of the core or the membrane by using different methods, said method being dependent on the stability of the substance. For example, the substance can be homogeneously mixed in the polymer matrix, or the polymer material and said substance can be dissolved in a suitable solvent or a mixture of solvents (dichloromethane, tetrahydrofuran etc.), removing most of the solvent under reduced pressure, letting the viscous solution to crystallize followed by further drying and granulating the drug-polymer composition. The therapeutically active substance can also be mixed into molten polymer, especially when thermoplastic elastomers are used, followed by cooling the mixture. Then the drug-polymer composition is processed to the desired shape by using known methods, for example such as moulding, injection moulding, rotation/injection moulding, casting, extrusion, such as co-extrusion, coating extrusion and/or blend-extrusion and other appropriate methods.

The material for the membrane, with or without any therapeutically active substance can be manufactured according to methods described above. The membrane can be assembled onto the cores, for example by moulding, spraying or dipping, or by using coating extrusion or coextrusion methods, or by mechanical stretching or expanding a prefabricated, tube formed membrane by pressurised gas, e.g. by air, or by swelling in a suitable solvent, for example such as propanol, isopropanol, cyclohexane, diglyme or the like.

The polymer rod thus obtained can be cut into pieces of the required length to form a compartment comprising a core or a core encased by a membrane. The compartment, or two or more compartments joined together, can be used as a subcutaneous implant, or attached to the body of an intrauterine device, or assembled to, for example, a substantially ring-shaped dosage form in any manner suitable for this purpose. The term “substantially ring-shaped” should be understood to encompass in addition to ring shaped dosage forms any other essentially ring-shaped structures that are appropriate for intrauterine or vaginal administration, such as for example helically coiled spirals and ring systems having convoluted surface. Intra-uterine devices may, in addition to a substantially ring-formed shape, have various other forms and may be for example T-, S-, 7- or omega-shaped. The compartment to be attached to an intrauterine device may be hollow so that it can be easily positioned over the body. Alternatively, the core can first be applied onto the body and in the next step be encased by a membrane. Implants have usually a rod-shaped form.

The ends of the compartments or the combination of compartments can be joined by using a coupling means which can be any method, mechanism, device or material known in the art for bonding or joining materials or structures together. The coupling can for example include solvent bonding, adhesive joining, heat fusing, heat bonding, pressure, and the like. Tubular compartments can also be joined by using a plug or a stopper made of any inert, biocompatible material, for example an inert material which does not permit the transport of active material. Further, substantially ring-shaped dosage forms can also be manufactured by placing a compartment or a combination of compartments in a mould at an elevated temperature and injecting molten high density polyethylene in between the ends, whereafter the prepared ring is cooled, or by joining the ends together by welding.

### Example 1

### Determination of the Inventive Gestagen Dose by Means of an Ovulation Inhibition Study

In an ovulation inhibition study the envisaged gestagen will be tested in various dosages to determine the gestagen effect on ovarian follicle maturation and ovulation with means of transvaginal ultrasound investigations and measurements of blood hormone levels (estradiol, progesterone). Furthermore, the cervical mucus will be investigated according to the Insler score with regard to intended changes of mucus characteristics typical for gestagen-only contraceptive methods (Insler V et al, Int J Gynecol Obstet 1972, 10(6): 223-228). The dose which inhibits ovulation below 95% and preferably in a range of approx. 40-80% and yields an Insler score of the cervical mucus of <9 will be chosen as gestagen dose in this invention. This dose will be specific for every gestagen. It is known to an expert in the field and therefore expected that some follicular growth will occur with this contraceptive method (e.g. occurrence of persistent ovarian follicles is a known effect of the gestagen pill Microlut®; see Fachinformation Microlut dated July 2007, page 2 [4.4.2 Warnhinweise; persistierende Ovarialfollikel]). Pharmacokinetic accumulation phenomena should be considered when identifying the dose.

### Example 2

### Effects of an Aromatase Inhibitor on Pituitary-Ovarian-Axis and Follicular Development

In a further pharmacodynamic study the effect of the AI applied via a parenteral dosage form, preferably an IVR, on the pituitary-ovarian-axis and follicular development will be investigated by determination of blood hormone levels (follicle stimulating hormone=FSH, luteinizing hormone=LH, estradiol, progesterone) and transvaginal ultrasound measurements alone and/or in combination with a gestagen. The lowest exposure to AI and gestagen which induces additional follicular growth as compared to the untreated or gestagen-treated cycle may serve as threshold for dosing of the AI in combination with gestagen. This dose will be specific for every AI. In the literature it is described that ovarian stimulation by an AI can occur at dosages of e.g. 2.5 mg Letrozole or 1 mg Anastrozole applied orally (Mitwally M F & Casper R F, Fertil Steril. 2001, 75(2):305-9, Fisher S A et al, Fertil Steril 2002 August; 78(2): 280-5, Badawy A et al, Fertil Steril 2008, 89(5): 1209-1212, Wu H H et al, Gynecol Endocrinol 2007, 23(2): 76-81). The targeted mean daily exposure, e.g. for Anastrozole delivered via the preferred parenteral dosage form which as said is an IVR or an IUD for this invention will be below 1 mg (or between 0.1 mg and 0.9 mg). For letrozole it will be below 2.5 mg (or between 0.1 mg and 2.4 mg).

The highest possible amount of AI combined with the gestagen in the dose described above will be determined by means of the human pharmacodynamic study described above which does not lead to additional stimulation of follicular growth compared to the gestagen alone as defined above. The gestagen effect on cervical mucus has to be maintained in the combination with AIs.

The experimental setup is valid for any parenteral application. For an IVR the above described experiments for the single components and for the combination would be performed with IVRs.

### Example 3

### Production of the Intravaginal Rings for the In Vivo Study

For an in vivo study with cynomolgus monkeys, anastrozole-releasing intravaginal rings adapted to the size of the cynomolgus monkeys were manufactured. The rings had an outer diameter of 14 mm and a cross-section of 2.3 mm.

The rings contained a core of anastrozole and elastomer, which core was coated by a release-controlling membrane. The intended drug dosages were achieved by appropriate selection of the materials for the core and the membrane and by adjusting the drug concentration and the surface of the anastrozole-containing core in combination with the membrane thickness. Suitable selection of these parameters makes it possible to control the release of anastrozole over periods of more than 30 days.

Three formulations (A, B, C; referred to as high, medium and low dose in FIG. 1) of anastrozole-releasing rings were produced, with each releasing anastrozole for at least 30 days. The starting dosages of anastrozole were 390 μg/day (A), 85 μg/day (B) or 27 μg/day (C). Placebo rings were likewise produced.

### a) Production of the Anastrozole-Releasing Rings

### Core

Two core compositions were prepared, with one containing anastrozole in a matrix made of silicone elastomer (polydimethylsiloxane) and the other containing only the silicone elastomer (polydimethylsiloxane). The anastrozole-containing core was produced by mixing (micronized) anastrozole and the silicone elastomer in a mixer. The anastrozole content of the mixture was 35% by weight. The mixture was shaped in a mold to give a small elastic rod having a thickness of 2 mm and cured (it would also have been possible to achieve this by extrusion through a nozzle). The silicone elastomer core was extruded to give a small elastic rod having a thickness of 2 mm (it would also have been possible to achieve this in a mold).

### Membrane

The drug-release-controlling membrane tube was produced from silicone elastomer (polydimethylsiloxane) by tube extrusion. The wall thickness of the tube (the membrane thickness) was about 1.5 mm.

### Assembly of the Ring

The anastrozole core was cut into three lengths: 38 mm (A), 6 mm (B) and 1.5 mm (C). The silicone elastomer core was cut into two lengths so that a total core length of 38 mm was achieved. The membrane tube was cut to a length of 38 mm and swollen in cyclohexane.

The ring was put together by pushing the core segment(s) into the swollen membrane tube. The tube was shaped into a ring by overlapping. After evaporation of the solvent, the tube contracted and compressed the parts tightly.

### Anastrozole Release

### Method

The release of anastrozole from the rings was analyzed in vitro at 37° C. in a 1% aqueous solution of 2-HP-β-CD (2-hydroxypropyl-beta-cyclodextrin) in a shaking bath (100 rotations/min). The solutions were changed daily except at the weekends. The sample solutions were analyzed by HPLC, using an Inertsil ODS-3, 150×4 mm 5 μm column and methanol/water (1/1) as eluent at a flow rate of 1.0 ml/min. The detection wavelength for anastrozole was 215 mm. Three rings were tested in parallel.

### In Vitro Release Rate

The rings were tested in vitro for up to 40 days. The in vitro release rate was continuous and controlled, but showed in the tests a reduction in the starting value of altogether about 30% after 30 days. The starting release rates were 390 μg/day (A), 85 μg/day (B) and 27 μg/day (C), and the mean release during the 30 days was 305 μg/day (A), 64 μg/day (B) and 16 μg/day (C).

The in vitro release rate of anastrozole is depicted in FIG. 1.

### Ex Vivo Study of the Primate Rings

The used rings (5) of the respective doses (A, B and C) were recovered and analyzed for residual anastrozole content. Anastrozole content was determined by extracting the ring with (THF), followed by HPLC analyses.

An estimated value for the release of anastrozole in vivo was obtained by calculating the reduction in the amount of anastrozole in the ring during use, e.g. the original content minus the ex vivo residual content, and dividing this by the number of days for which the ring was in use (varied). Table 1 lists the average (5 rings) ex vivo anastrozole content per dose and the anastrozole content in the comparative rings (unused rings) along with the calculated average anastrozole release rate per day.

TABLE 1
Estimated value for the in vivo anastrozole release per day for doses A, B
and C, calculated from the average in vivo test duration and the average
assay results for the ex vivo rings and the unused comparative rings

| | | Average assay | Average |
|---|---|---|---|
| | Average assay | value for the | release |
| | value for the ex | comparative rings | rate per day |
| Dose | vivo rings (mg) | (mg) | (μg/day) |
| A | 32.8 | 41.1 | 277 |
| B | 4.9 | 6.5 | 54 |
| C | 1.1 | 1.5 | 15 |

### Example 4

### Demonstration of Feasibility in Cynomolgus Monkeys

The cynomolgus monkey is suitable as an animal model for studying aspects of human endocrinology because its reproductive system is comparable to that of humans (Weinbauer, N., Niehaus, Srivastav, Fuch, Esch, and J. Mark Cline (2008). “Physiology and Endocrinology of the Ovarian Cycle in Macaques.” Toxicologic Pathology 36(7): 7S-23S). This comprises, among other things, cycle length, hormone receptors, morphology, endocrine system and regulation of the pituitary-ovarian axis (Borghi, M. R., R. Niesvisky, et al. (1983). “Administration of agonistic and antagonistic analogues of LH-RH induce anovulation in Macaca fasicularis.” Contraception 27(6): 619-626. Satoru Oneda, T. I., Katsumi Hamana (1996). “Ovarian Response to Exogenous Gonadotropins in Infant Cynomolgus Monkeys” International Journal of Toxicology, 15(3): 194-204). The pharmacodynamic and pharmacokinetic effect of intravaginally administered dosages of the aromatase inhibitor anastrozole was studied over the duration of a menstrual cycle by inserting a vaginal ring (IVR) having three different release rates. Among other things, the influence on the pituitary-ovarian axis was studied by determining the hormones estradiol, FSH, progesterone (the blood collections required for this were carried out over the entire experimental period; on day 1, four collections [0 h, 1 h, 3 h, 6 h after insertion of the IVR]; 1 collection each on days 2 and 3; after this time point, further collections followed on every 3rd day) and by ultrasound scans of the ovary (2× per week). Hormone determination was carried out according to the instructions provided by the supplier (estradiol [Siemens/DPC], progesterone [Beckmann-Culter/DSL], FSH [SHG]). An IVR having an initial in vitro release of 0 μg/day (placebo, no anastrozole), 390 μg/day, 85 μg/day or 27 μg/day was inserted into five animals per group one to three days after the last day of menstruation. Animals having irregular cycles were excluded from the experiment.

A reduction in estradiol levels over the entire cycle with a significant fall during the follicular phase—important for the estrogen-dependent proliferation of the endometrium and endometriotic lesions—was observed in the group having an initial release of 390 μg/day (table 2, row 5 and FIG. 2). As shown in rows 1, 2 and 3 of table 2, counterregulation by the pituitary-ovarian axis fails to occur at the dosages used (no difference compared to the placebo control). Comparable FSH levels among the groups show that the dosages used have no stimulatory effect on the pituitary-ovarian axis. In agreement with this observation, no formation of ovarian cysts was observed (cf. row 7, table 2). This experiment shows that it is possible in an animal model to lower endogenous estrogen levels using an aromatase inhibitor (for example, anastrozole) without triggering counterregulation.

The following tables contain a summary of the in vivo and in vitro release rates [table 1] of anastrozole from the IVR, the levels of estradiol (E2), progesterone and FSH with different dosages of anastrozole, and information about the formation of ovarian cysts during the cycle (days 1-26) [table 2].

TABLE 1
Summary of the in vivo and in vitro release rates

| | Anastrozole |
|---|---|
| Initial (day 1) in vitro release (μg/day) | |
| (A) | 390 |
| (B) | 85 |
| (C) | 27 |
| Average (30 days) in vitro release | |
| (μg/day) | |
| (A) | 305 |
| (B) | 64 |
| (C) | 16 |
| Average (30 days) in vivo serum | |
| concentration (μg/l) | |
| (A) | 5.9 |
| (B) | 1.4 |
| (C) | 0.3 |
| Average (30 days) in vivo release | |
| (μg/day) (PC-based) | |
| (A) | 278 |
| (B) | 66 |
| (C) | 16 |
| Based on the ex vivo IVR analysis | |
| (A) | 277 |
| (B) | 54 |
| (C) | 15 |
| Plasma protin binding [free fraction, | |
| fu] | |
| Cynomolgus monkey | 34% |
| Human | 52% |
| CLₚₗ [l/h/kg] | |
| Cynomolgus monkey | 0.58 |
| Human (CL/F) | 0.02 |
| Calculated constant in vivo IVR release | ≈250 μg/day/60 kg patient |
| rate in humans (to maintain plasma | |
| levels which correspond to those of the | |
| effective dose in cynomolgus monkeys) | |
| Calculated constant in vitro IVR release | ≈270 μg/day/60 kg patient |
| rate (in buffer) (to maintain in humans | |
| plasma levels which correspond to those | |
| of the effective dose in cynomolgus | |
| monkeys) | |
| Calculated initial in vitro IVR release | ≈350 μg/day/60 kg patient |
| rate (in buffer) with a decreasing release | |
| rate (32% in 4 weeks) (to maintain in | |
| humans plasma levels which correspond | |
| to those of the effective dose in | |
| cynomolgus monkeys) (human dose) | |

TABLE 2
Estradiol (E2), progesterone and FSH levels and the formation of ovarian cysts during the cycle (days 1-26).

| | | | Anastrozole | Anastrozole | Anastrozole | |
|---|---|---|---|---|---|---|
| | | | 27 μg/day | 85 μg/day | 390 μg/day | |
| | | | (initial in | (initial in | (initial in | P value vs |
| | | Placebo | vitro release) | vitro release) | vitro release) | placebo |
| 1 | FSH (μg/l) | 4.85 +/− 2.70 | 5.52 +/− 3.07 | 4.90 +/− 2.58 | 4.83 +/− 2.91 | Not signif- |
| | Mean level/day without | | | | | icant |
| | preovulatory maximum | | | | | |
| 2 | Progesterone (nmol/l) | 5.65 +/− 5.99 | 5.57 +/− 5.11 | 6.58 +/− 3.91 | 4.58 +/− 2.64 | Not signif- |
| | Mean level/day | | | | | icant |
| | Follicular phase | | | | | |
| 3 | Progesterone (nmol/l) | 51.61 +/− 37.54 | 91.92 +/− 52.78 | 60.02 +/− 22.65 | 92.88 +/− 55.50 | Not signif- |
| | Mean level/day | | | | | icant |
| | Luteal phase | | | | | |
| 4 | E2 pmol/l | 3768 +/− 684.9 | 4862 +/− 1986 | 4126 +/− 2063 | 2784 +/− 999.8 | Not signif- |
| | AUC (cycle days 1-26) | | | | | icant |
| 5 | E2 pmol/l/day | 3137 +/− 295.5 | 3854 +/− 927.5 | 3235 +/− 1101 | 1978 +/−350.6 | P < 0.0478 |
| | AUC (follicular, cycle | | | | | (anastrozole |
| | days 1-17) | | | | | 390 μg/day |
| | | | | | | vs placebo) |
| 6 | E2 pmol/l | 404 +/− 211.9 | 403.2 +/− 169.7 | 605.1 +/− 264.1 | 342.9 +/− 135.2 | Not signif- |
| | AUC (luteal, cycle | | | | | icant |
| | days 17-26) | | | | | |
| 7 | Ovarian cysts | None | None | None | None | N.A |
| | (ultrasound) | | | | | |

FIG. 2 shows the estradiol levels (pmol/l) during the follicular phase. 390 μg of anastrozole per day lowers the estradiol levels significantly (P value<0.0478) compared to the placebo group.

The concentration of anastrozole in plasma samples was quantitatively determined by means of liquid-liquid extraction with liquid chromatography coupled to tandem mass spectrometry (LC/ESI-MS/MS). The analyses were carried out on an Agilent 1200 and an AB Sciex Triple Quad 5500 in positive ionization mode. For this purpose, 100 μl were initially taken from each plasma sample, admixed with 300 μl of an aqueous solution containing any non-structurally-related compound as internal standard, and extracted with 1.3 ml of methyl tert-butyl ether on a Perkin Elmer Mass Prep Station. After phase separation, the organic phase was blown off and the residue was absorbed with 30 μl of LC eluent (50% methanol/50% water, v/v). 5 μl of this were injected into the LC/MS/MS, the m/z transition 294 ([M+H]+)→225 was recorded, and the signal was integrated with the AB Sciex Software Analyst 1.5. The concentrations of the plasma samples were determined from the resulting areas with the aid of a calibration curve present in the same sequence (0, 0.0500 to 1000 nM in plasma, n=2). The lower limit of determination of this method was about 1.2 μg/l (quadratic calibration curve, weighting 1/x). The time courses for the serum concentration of anastrozole can be found in FIG. 3. Plasma protein binding (free fraction [fu]) of anastrozole in human and cynomolgus monkey plasma was determined by means of equilibrium dialysis (cf. Banker, M. J. Banker, et al. (2003). “Development and Validation of a 96-Well Equilibrium Dialysis Apparatus for Measuring Plasma Protein Binding” J. Pharma. Sci. 92(5): 967-974) over seven hours at 37° C., in a 96-well based microdialysis apparatus (HT-Dialysis LLC) with a dialysis membrane made of regenerated cellulose (MWCO 3.5K) and subsequent measurement of the dialysate by means of LC/ESI-MS/MS. Calculation of the free fraction (fu) yielded 34% in humans and 52% in the cynomolgus monkey.

FIG. 3 shows the time courses for the plasma concentration of anastrozole after IVR administration in female cynomolgus monkeys.

The mean plasma concentration (Css) of anastrozole was calculated as the mean value of all measured concentrations per dose group from the day after insertion of the IVR up to the end of the experiment.

To calculate the in vivo release rate of anastrozole from the vaginal ring, the in vivo plasma clearance (CL) in female cynomolgus monkeys was determined in a separate experiment. For this experiment, anastrozole was intravenously administered to female cynomolgus monkeys at a dose of 0.2 mg/kg in 50% PEG400 in each case, blood samples were taken at different times, and the plasma concentration was determined by means of LC/ESI-MS/MS. The plasma clearance (CL) thus calculated was 0.58 l/h/kg for anastrozole.

The mean in vivo release rates (Rin) from the IVR were subsequently calculated according to the equation: Rin=Css*CL (see table X). It became apparent that the mean release rates calculated in this way were a good match for the in vitro release rates in buffer (in vitro/in vivo correction factor of 1.1). Furthermore, they were in good agreement with the mean in vivo release rate calculated from the ex vivo residual content of the used rings at the end of the study.

Subsequently, an estimation was made of the in vitro IVR release rate of an IVR for human application, which is necessary to achieve serum levels which led to a lowering of estradiol in the monkeys. In the cynomolgus monkeys, this was achieved in the highest dose group at a mean serum concentration (Css) of 5.9 μg/l. The corresponding effective serum concentration in humans is estimated to be 9 μg/l, taking into account species-specific plasma protein binding, according to equation (1) below.

$${Css}_{human} = {Css}_{monkey} \cdot \frac{{fu}_{monkey}}{{fu}_{human}} \qquad Equation 1$$

The mean in vivo release rate from the IVR which is necessary to achieve a plasma concentration of 9 μg/l in humans is calculated according to equation 2. For this equation, the plasma clearance of anastrozole in humans is required. This is known only for oral administration (CL/F) (Clin. Pharmacol. and Biopharmac. Review, NDA 020541 (Sep. 28, 1995)) and was able to be used as the CL for the calculation, since the oral bioavailability (F) is approximately 1.

Rinₕᵤₘₐₙ=Cssₕᵤₘₐₙ·CLₕᵤₘₐₙ Equation 2:

A human in vivo release rate of 246 μg/d was obtained which has to be kept constant in order to achieve levels in humans which achieved a lowering of estradiol in the monkeys. Assuming comparable permeation of anastrozole in the vagina of primates and humans, the in vitro/in vivo correction factor of 1.1 calculated from the primate experiment gives, for humans, a constant in vitro release rate in buffer of 270 μg of anastrozole/d. If, for the IVR in humans, there is a comparable fall in the release rate over time, as for the monkeys, the corresponding initial in vitro release rate would need to be higher; this was calculated to be about 350 μg per day (table 1).

### LIST OF FIGURES

FIG. 1: In vitro release rate (μg/d) of anastrozole for formulations A (high dose=390 μg/day), B (medium dose=85 μg/day) and C (low dose=27 μg/day)

FIG. 2: Estradiol levels (pmol/1) during the follicular phase. 390 μg of anastrozole per day lowers the estradiol levels significantly (P value <0.0478) compared to the placebo group.

FIG. 3: Time courses for the plasma concentration of anastrozole after IVR administration in female cynomolgus monkeys

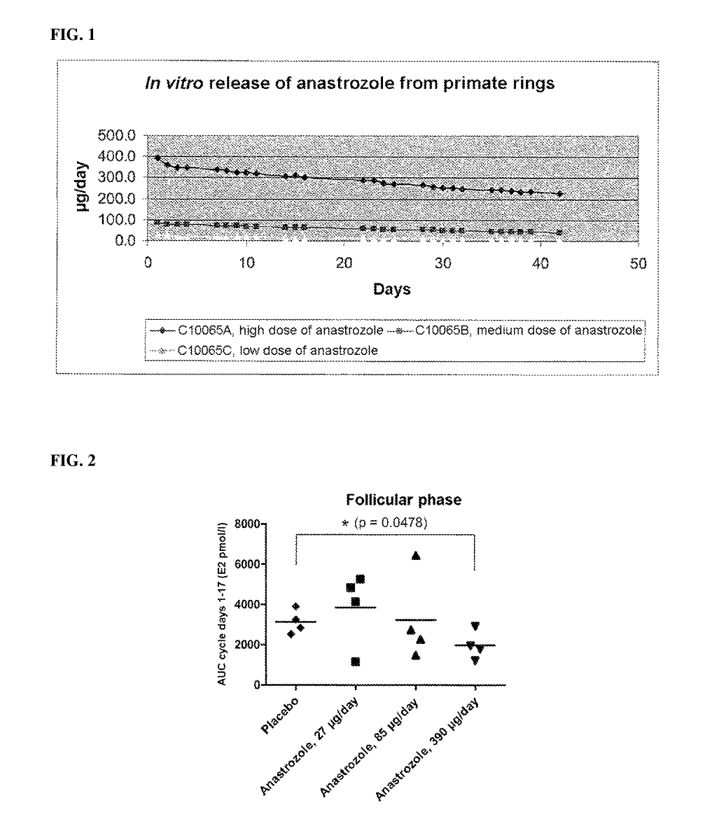

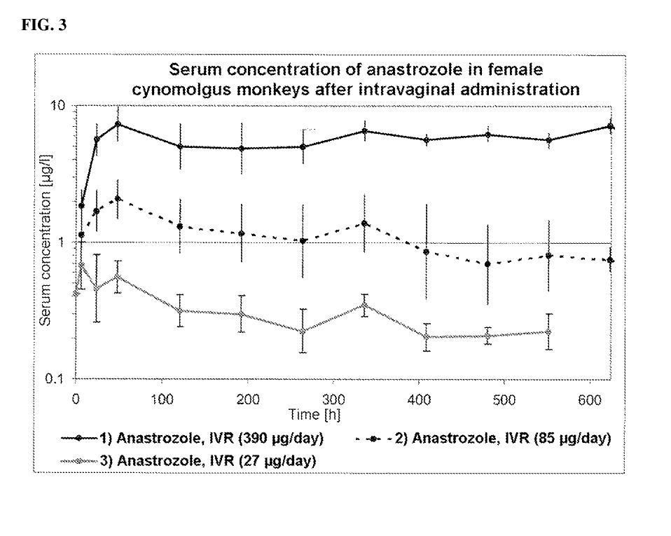

1-9. (canceled)

10. The IVR of claim 20, wherein the desired release rates claimed therein the systemic anastrozole and levonorgestrel levels are achieved only one, two or three days after the start of treatment owing to a burst effect.

11-14. (canceled)

15. An IVR of the dosage form of claim 20 in which the long-term release period lasts from 1 week to 3 months.

16. An IVR of the dosage form of claim 20 in which the long term release period lasts from 4 to 6 weeks.

17-19. (canceled)

20. An intravaginal ring (IVR) comprising anastrozole and levonorgestrel, the IVR adapted to treat endometriosis by achieving after insertion into a patient a systemic anastrozole level that corresponds to a daily oral administration of less than 1 mg of anastrolzole and a systemic levonorgestrel level that corresponds to a daily oral administration of between 10 μg and 50 μg of levonorgestrel, and wherein the IVR does not comprise an estrogen or ethinyl estradiol.

21. The IVR of claim 20, wherein the systemic anastrozole level corresponds to a daily oral administration of between 0.1 mg and 0.9 mg of anastrozole
